(19) United States
(12) Patent Application Publication
(10) Pub. No.: US 2020/0375992 A1
(43) Pub. Date: Dec. 3, 2020

(54) PHARMACEUTICAL COMPOSITION AND USE FOR APPLYING PALBOCICLIB IN DISEASE TREATMENT OF PATIENT AND INHIBITION OF PI3K ACTIVITY

(71) Applicant: Chang Gung University, Taoyuan City (TW)

(72) Inventors: Tsong-Long HWANG, Taoyuan City (TW); Po-Jen CHEN, Taoyuan City (TW); Hsin-Hui TSENG, Taoyuan City (TW)

(21) Appl. No.: 16/793,240

(22) Filed: Feb. 18, 2020

(30) Foreign Application Priority Data
May 29, 2019 (TW) 108118624

(51) Int. Cl. A61K 31/519; A61P 17/06

(52) U.S. Cl. CPC A61K 31/519; A61P 17/06

(57) ABSTRACT

The invention provides a pharmaceutical composition for treating disease in a patient and inhibition of PI3K activity. The pharmaceutical composition includes an effective amount of Palbociclib and a pharmaceutically acceptable carrier. The invention further provides a pharmaceutical composition for use in the treatment of a disease in a patient. The application of the pharmaceutical composition of the present invention and use thereof are advantageous for inhibiting of PI3K activity and thus treating a disease.

### CROSS REFERENCE TO RELATED APPLICATIONS

This Non-provisional application claims priority under 35 U.S.C. § 119(a) on Patent Application No(s). 108118624 filed in Taiwan, Republic of China on May 29, 2019, the entire contents of which are hereby incorporated by reference.

### BACKGROUND OF THE INVENTION

### Field of Invention

The invention relates to a pharmaceutical composition for treating a patient with a disease and inhibiting PI3K activity and use thereof.

### Related Art

Phosphatidylinositol-4,5-bisphosphate 3-kinase (PI3K) is a member in the lipid kinase family which can phosphorylate phosphatidylinositol (PI) or the 3 position hydroxyl group (3′-OH) of phosphatidylinositol. PI3K is a critical enzyme belonging to signal transduction pathways which transducing a signal from cell surface receptor to downstream effectors. The PI3K family includes at least 15 different enzymes divided by structural homology. They are divided into three classes based on sequence homology and products formed by their catalyzes. Class I PI3Ks may phosphorylate phosphatidylinositol (PI), phosphatidylinositol-4-phosphate (PI4P), and phosphatidylinositol (4,5)-bisphosphate (PI(4,5)P2) to generate phosphatidylinositol-3-phosphate (PI3P), phosphatidylinositol-3,4-bisphosphate (PI(3,4)P2), and phosphatidylinositol (3,4,5)-trisphosphate (PIP3), respectively. Class II PI3Ks may phosphorylate PI and PI4P. Class III PI3Ks merely phosphorylate PI.

Currently, four different types of class I PI3K have been identified, namely PI3K-α, PI3K-β, and PI3K-ω, respectively. Each of them is composed of different p110 catalytic subunits and regulatory subunits. In general, class I PI3Ks are activated by tyrosine kinase receptors or G protein-coupled receptors to generate PIP3. PIP3 binds to some downstream effectors, such as the effectors in Akt/PDK1 pathway, mTOR, Tec kinase family and GTPase of Rho family.

PI3K-δ of class I PI3K is involved in mammalian immune system functions, such as T cell function, B cell activation, mast cell activation, dendritic cell function, and neutrophil activity. PI3K-δ is involved in many diseases related to immune response, such as allergic reactions, inflammatory diseases, autoimmune diseases (such as systemic lupus erythematosus) asthma, emphysema, and other respiratory diseases, due to its role in the function of the immune system.

Accordingly, it is an urgent need to provide a pharmaceutical composition for inhibiting PI3K activity and use thereof. The pharmaceutical composition can reduce PI3K activity in a patient for avoiding the effect of PI3K to the function of the immune system, and thus achieves the efficacy of treating immune-related diseases.

### SUMMARY OF THE INVENTION

In view of the foregoing objectives, a purpose of the invention is to provide a pharmaceutical composition for inhibiting PI3K activity and use thereof. The pharmaceutical composition can reduce PI3K activity in an individual for avoiding the effect of PI3K to the function of the immune system, and thus achieves the efficacy of treating immune-related diseases.

To achieve the above objective, the invention provides a pharmaceutical composition for treating a disease in a patient. The pharmaceutical composition comprises an effective amount of Palbociclib and a pharmaceutically acceptable carrier, wherein Palbociclib is used to treat the disease by inhibiting phosphatidylinositol-4,5-bisphosphate 3-kinase (PI3K) activity.

To achieve the above objective, the invention also provides a pharmaceutical composition for use in the treatment of a disease in a patient, wherein the pharmaceutical composition comprises an effective amount of Palbociclib and a pharmaceutically acceptable carrier. Palbociclib is used to treat the disease by inhibiting PI3K activity.

To achieve the above objective, the invention further provides a pharmaceutical composition for inhibiting PI3K activity. The pharmaceutical composition comprises an effective amount of Palbociclib and a pharmaceutically acceptable carrier.

To achieve the above objective, the invention further provides a method for treating a disease in a patient, the method comprises a step of providing a pharmaceutical composition to the patient. The pharmaceutical composition comprises an effective amount of Palbociclib and a pharmaceutically acceptable carrier, wherein Palbociclib is used to treat the disease by inhibiting PI3K activity.

To achieve the above objective, the invention further provides a method for inhibiting PI3K activity in a patient, the method comprises a step of providing a pharmaceutical composition to the patient. The pharmaceutical composition comprises an effective amount of Palbociclib and a pharmaceutically acceptable carrier.

In one embodiment, the disease is acute lung injury, acute respiratory distress syndrome, psoriasis, chronic obstructive pulmonary disease, pulmonary fibrosis, liver injury, fatty liver disease, liver fibrosis, myocardial infarction, shock, stroke, vasculitis, sepsis, inflammatory bowel disease, systemic lupus erythematosus or atopic dermatitis.

In one embodiment, Palbociclib is used to treat the disease by inhibiting neutrophils activity.

In one embodiment, the effective amount of Palbociclib is from 1 μg/kg of the body weight/per day to 100 mg/kg of the body weight/per day.

As mentioned above, the efficacy of this invention is to provide a pharmaceutical composition for inhibiting PI3K activity and use thereof The pharmaceutical composition can reduce PI3K activity in a patient for avoiding the effect of PI3K to the function of the immune system, and thus achieves the efficacy of treating diseases.

### BRIEF DESCRIPTION OF THE DRAWINGS

FIGS. 1A-1C show results of PI3K activity inhibited by Palbociclib. FIG. 1A shows a result of PI3K-α activity inhibited by the different amount of Palbociclib. FIG. 1B shows a result of PI3K-β activity inhibited by the different amount of Palbociclib. FIG. 1C shows a result of PI3K-δ activity inhibited by the different amount of Palbociclib.

FIGS. 2A-2B show results that Palbociclib restricts superoxide anion generation and elastase release in the formyl-Met-Leu-Phe (fMLF)-stimulated neutrophils. FIG. 2A shows a result that Palbociclib significantly restricts superoxide anion generation in the fMLF-stimulated neutrophils. FIG. 2B shows a result that Palbociclib significantly restricts elastase release in the fMLF-stimulated neutrophils.

FIGS. 3A-3B show results that Palbociclib reduce the psoriasis-like symptoms in imiquimod (IMQ)-treated mice. FIG. 3A shows the photos of mice skin on the 5^{th} day after the different treatments. FIG. 3B shows the partially enlarged photos of mice skin after 0 to 5^{th} day treated by different treatments.

### DETAILED DESCRIPTION OF THE INVENTION

The embodiments and examples of the pharmaceutical composition for inhibiting PI3K activity and the use thereof in this invention will be apparent from the following detailed description, which proceeds with reference to the accompanying figures, wherein the same references relate to the same elements.

The pharmaceutical composition of this invention and use thereof can reduce PI3K activity in a patient for avoiding the effect of PI3K to the function of the immune system, and thus achieves the efficacy of treating diseases.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used. It is also to be understood that the terminology used herein is for the purpose of describing particular embodiments only, and is not intended to be limiting.

As used herein, the terms “phosphatidylinositol-4,5-bisphosphate 3-kinase” and “PI3K” refer to a member in the lipid kinase family which can phosphorylate phosphatidylinositol (PI) or the 3′-OH group of PI. PI3K is a critical enzyme belonging to signal transduction pathways which transducing a signal from cell surface receptor to a downstream effector. The PI3K family includes at least 15 different enzymes divided by structural homology. They are divided into three classes based on sequence homology and products formed by their catalyzes. Class I PI3Ks may phosphorylate PI, PI4P, and PI(4,5)P2 to generate PI3P, PI(3,4)P2, and PIP3, respectively. Class II PI3Ks may phosphorylate PI and PI4P. Class III PI3Ks merely phosphorylate PI. Four different types of class I PI3K have been identified, namely PI3K-α, PI3K-β, PI3K-δ, and PI3K-ω, respectively. Each of them is composed of different p110 catalytic subunits and regulatory subunits. Class I PI3K (especially PI3K-δ) is associated with immune-related diseases.

The term “Palbociclib” is also called “PD-0332991”, “PD0332991”, “PD 0332991”, “Ibrance” and “571190-30-2”, which is a useful CDK4 and CDK6 selective inhibitor. The IUPAC name of Palbociclib is “6-acetyl-8-cyclopentyl-5-methyl-2-((5-(piperazin-1-yl)pyridin-2-yl)amino)pyrido [2,3-d]pyrimidin-7(8H)-one”. In the USA, Palbociclib is approved for treating hormone receptor (HR)-positive and human epidermal growth factor receptor 2 (HER2)-negative breast cancer, advanced breast cancer or metastatic breast cancer. Palbociclib is combined with letrozole as initial endocrine therapy or combined with fulvestrant after the progression of disease in endocrine therapy. The drug is marketed by Pfizer under the trade name IBRANCE® in a dosage form of capsule containing 100 mg of Palbociclib. Palbociclib of this invention can includes the following structure,

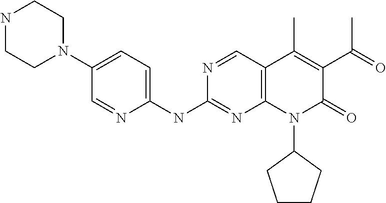

or salt, solvate, hydrate, prodrug, enantiomer, diastereoisomer, or tautomer thereof

As used herein, a “disease” is a state of health of a subject wherein the subject cannot maintain homeostasis, and wherein if the disease is not ameliorated then the subject's health continues to deteriorate.

The terms “treat”, “treating” and “treatment” as used herein, means reducing the frequency or severity with which symptoms of a disease or condition are experienced by a subject by virtue of administering an agent or pharmaceutical composition to the subject.

As used herein, the term “pharmaceutically acceptable” refers to a material, such as a carrier or diluent, which does not abrogate the biological activity or properties of Palbociclib, or salt, solvate, hydrate, prodrug, enantiomer, diastereoisomer, or tautomer thereof useful within the invention, and is relatively non-toxic, i.e., Palbociclib may be administered to a subject without causing undesirable biological effects or interacting in a deleterious manner with any of the components of the composition in which it is contained.

As used herein, the term “pharmaceutically acceptable carrier” means a pharmaceutically acceptable salts, material, composition or carrier, such as a liquid or solid filler, diluent, excipient, solvent or encapsulating material, involved in carrying or transporting Palbociclib of the invention within or to the subject such that Palbociclib may perform its intended function. Typically, Palbociclib is carried or transported from one organ or portion of the body to another organ or portion of the body. Each salt or carrier must be compatible with the other ingredients of the formulation, including Palbociclib useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agents; lubricating agent; binding agents; disintegrating agents; wetting agents; emulsifying agents; coloring substances; releasing agents; coating agents; sweetening agents; flavoring agents; aromatic agents; preservatives; antioxidants; plasticizers; gelling agents; thickening agents; hardening agents; setting agents; suspending agents; surface active agents; humectant; carriers; stabilizers; and other non-toxic compatible substances employed in pharmaceutical formulations.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous or another route of administration. Other contemplated formulations include projected nanoparticles, microspheres, liposomal preparations, coated particles, polymer conjugates, resealed erythrocytes containing the active ingredient, and immunologically-based formulations.

Suitable pharmaceutical compositions and dosage forms include, for example, tablets, capsules, caplets, pills, gel caps, troches, emulsions, dispersions, suspensions, solutions, syrups, granules, beads, transdermal patches, gels, powders, pellets, magmas, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

The pharmaceutical composition of this invention can be administered orally to a patient in any orally acceptable dosage form, such as, but not limited to, capsules, tablets, aqueous suspensions or solutions. In the case of tablets for oral use, commonly used carriers include lactose and corn starch. Alternatively, a lubricant such as magnesium stearate may be added to the pharmaceutical composition. For oral administration in capsule form, useful diluents include lactose and dried corn starch. In the case of aqueous suspensions for oral use, the active ingredient “Palbociclib” is combined with emulsifying agents and suspending agents. In other embodiments, specific sweetening agents, flavoring agents or coloring agents may be added to the pharmaceutical composition for easily swallowed by a patient.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

Formulations suitable for topical administration include, but are not limited to, liquid or semi liquid preparations such as liniments, lotions, oil-in-water or water-in-oil emulsions such as creams, ointments or pastes. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein. The carrier in the ointment for topical administration may be, for example, but not limited to, mineral oil, liquid vaseline, propylene glycol, polyoxyethylene, polyoxypropylene compound, emulsion wax, or water.

A pharmaceutical composition of the invention may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those of skill in the art.

A pharmaceutical composition of the invention may be prepared, packaged, or sold in a formulation suitable for rectal administration. Such a composition may be in the form of, for example, a suppository, a retention enema preparation, and a solution for rectal or colonic irrigation. The pharmaceutical composition of the invention may further includes suitable and non-irritating excipients to prepare a suppository. The excipient is solid at ordinary room temperature and is liquid at the rectal temperature of the subject. Therefore, the suppository would dissolved in the rectal and releasing the active ingredient “Palbociclib”. Specifically, excipients include coconut cream, beeswax and polyethylene glycol.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, the terms “patient”, “individual” and “subject” can be used interchangeably and may refer to a human or non-human mammal. Non-human mammals include, for example, livestock and pets, such as ovine, bovine, porcine, canine, feline and murine mammals. Preferably, the subject is human.

As used herein, the terms “neutrophil”, “neutrophilic leukocyte” and “neutrocyte” can be used interchangeably and may refer to a major leukocyte in mammal blood. 60 to 70% of leukocytes are neutrophils. Neutrophils play a very important role in the innate immune system. The morphology of neutrophils is irregular and often has protrusions on the periphery of the neutrophils. The cell nucleus of immature neutrophils is band-shaped or horseshoe-shaped. The cell nucleus of mature neutrophils is segmented form, and the most common form is three segments. There are many steps in the procedure of the differentiation from the myelocyte to the neutrophil. First, the myeloblasts differentiate into the promyelocytes and start to produce primary granules (also known asazurophil). Second, the promyelocytes differentiate into myelocytes which are present in the bone marrow and start to produce secondary granules (also known as specific granules). The myelocytes further differentiate into the band cells and leaving the bone marrow to enter the blood circulation and start to produce tertiary granules (also known as gelatinase granules). Finally, the band cells differentiate into the neutrophils (also known as neutrophilic granulocyte) and start to produce secretory granules. Among the granules that can be produced by mature neutrophils, the largest granule is azurophil which is about 0.3 μm. There are many substances in the granules, including myeloperoxidase (MPO), serine protease (such as proteinase 3), cathepsin G, neutrophil elastase, lysozyme, and so on. The main function of serine proteases in the granules is to break down the pathogens in the phagosome and the lysosome. Serine proteases will also be secreted to the outside of the cell to destroy foreign pathogens. Neutrophils have strong chemotaxis and phagocytosis. As the phagocytosis began, the cell membrane is disturbed and respiratory outbreaks are caused. The oxygen consumption of the cells increases, and large amounts of cytotoxic effectors such as peroxides and superoxides are produced, which have killing activity against pathogens. In addition to using phagocytosis and secreted proteins to kill the pathogens, when the pathogens invade, neutrophils can release their own DNA to encapsulate pathogens. The said DNA is reticulated. The reticulated DNA and some enzymes originally attached to the cell nucleus then moved and attached to the DNA (for example, myeloperoxidase and neutrophil elastase) are used to encapsulate pathogens. At the same time, DNA attached to the pathogens by the negative charge of DNA. Finally, the enzyme is used to kill the pathogens. The aforementioned mechanism is called neutrophil extracellular traps (NETs). Thus, the myeloperoxidase and elastase which are produced by neutrophils are associated to the neutrophil activity. When neutrophils are over-activated, excessive oxidative stress, excessive release of granule substances, and formation of excessive NETs will damage cells or tissues. Therefore, over-activated of neutrophils is associated with many diseases, such as acute lung injury, acute respiratory distress syndrome, psoriasis, chronic obstructive pulmonary disease, pulmonary fibrosis, liver injury, fatty liver disease, liver fibrosis, myocardial infarction, shock, stroke, vasculitis, sepsis, inflammatory bowel disease, systemic lupus erythematosus or atopic dermatitis.

As used herein, the term “effective amount” refers to the dose of Palbociclib which can inhibit PI3K activity. In this invention, the amount of Palbociclib is from 1 μg/kg of the body weight/per day to 100 mg/kg of the body weight/per day.

Ranges: throughout this invention, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual and partial numbers within that range, for example, 1, 2, 2.7, 3, 4, 5, 5.3, and 6. This applies regardless of the breadth of the range.

A pharmaceutical composition of the invention is used for treating a disease in a patient. The pharmaceutical composition includes an effective amount of Palbociclib and a pharmaceutically acceptable carrier. Wherein Palbociclib is used to treat the disease by inhibiting PI3K activity. In this embodiment, an amount of Palbociclib can be taken or weighted, and then added to a pharmaceutically acceptable carrier to prepare a pharmaceutical composition. The pharmaceutical composition is administered to the patient to achieve the efficacy of inhibiting PI3K activity, and thus used for the treatment of a disease. In particular, Palbociclib can includes the following structure,

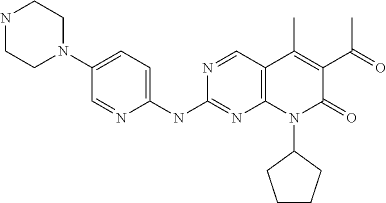

or salt, solvate, hydrate, prodrug, enantiomer, diastereoisomer, or tautomer thereof and does not pose a limitation of the invention.

In addition, pharmaceutical compositions of this invention include, for example, but not limited to tablets, capsules, caplets, pills, gel caps, troches, emulsions, dispersions, suspensions, solutions, syrups, granules, beads, transdermal patches, gels, powders, pellets, magmas, creams, pastes, plasters, lotions, discs, suppositories, liquid sprays for nasal or oral administration, dry powder or aerosolized formulations for inhalation, compositions and formulations for intravesical administration and the like.

In this embodiment, the types of the carriers are described above, and therefore is omitted here for conciseness.

In this embodiment, the disease is, for example, but not limited to acute lung injury, acute respiratory distress syndrome, psoriasis, chronic obstructive pulmonary disease, pulmonary fibrosis, liver injury, fatty liver disease, liver fibrosis, myocardial infarction, shock, stroke, vasculitis, sepsis, inflammatory bowel disease, systemic lupus erythematosus, atopic dermatitis or other diseases associated with PI3K, immune or neutrophils known by a person in the art.

In this embodiment, Palbociclib is used to treat the disease by inhibiting neutrophils activity.

In this embodiment, the effective amount of Palbociclib is from 1 μg/kg of the body weight/per day to 100 mg/kg of the body weight/per day. Preferably, the effective amount of Palbociclib is 1 μg, 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 50.5 mg, 51 mg, 51.5 mg, 52 mg, 52.5 mg, 53 mg, 53.5 mg, 54 mg, 54.5 mg, 55 mg, 55.5 mg, 56 mg, 56.5 mg, 57 mg, 57.5 mg, 58 mg, 58.5 mg, 59 mg, 59.5 mg, 60 mg, 60.5 mg, 61 mg, 61.5 mg, 62 mg, 62.5 mg, 63 mg, 63.5 mg, 64 mg, 64.5 mg, 65 mg, 65.5 mg, 66 mg, 66.5 mg, 67 mg, 67.5 mg, 68 mg, 68.5 mg, 69 mg, 69.5 mg, 70 mg, 70.5 mg, 71 mg, 71.5 mg, 72 mg, 72.5 mg, 73 mg, 73.5 mg, 74 mg, 74.5 mg, 75 mg, 75.5 mg, 76 mg, 76.5 mg, 77 mg, 77.5 mg, 78 mg, 78.5 mg, 79 mg, 79.5 mg, 80 mg, 80.5 mg, 81 mg, 81.5 mg, 82 mg, 82.5 mg, 83 mg, 83.5 mg, 84 mg, 84.5 mg, 85 mg, 85.5 mg, 86 mg, 86.5 mg, 87 mg, 87.5 mg, 88 mg, 88.5 mg, 89 mg, 89.5 mg, 90 mg, 90.5 mg, 91 mg, 91.5 mg, 92 mg, 92.5 mg, 93 mg, 93.5 mg, 94 mg, 94.5 mg, 95 mg, 95.5 mg, 96 mg, 96.5 mg, 97 mg, 97.5 mg, 98 mg, 98.5 mg, 99 mg, 99.5 mg or 100 mg/kg of the body weight/per day. Of course, the effective amount of Palbociclib may be any value and range encompassed between any two values within the foregoing ranges and may be changed according to the carrier which is used, the route of administration, or the individual who in need and the physiology state thereof

This invention also provides a pharmaceutical composition for use in the treatment of a patient. This invention further provides a pharmaceutical composition for inhibiting PI3K activity. This invention further provides a pharmaceutical composition for use in inhibiting PI3K activity. In addition, this invention provides a method for treating a disease in a patient. The method includes providing a pharmaceutical composition comprising an effective amount of Palbociclib and a pharmaceutically acceptable carrier. Palbociclib is used to treat the disease by inhibiting PI3K activity. The disease is acute lung injury, acute respiratory distress syndrome, psoriasis, chronic obstructive pulmonary disease, pulmonary fibrosis, liver injury, fatty liver disease, liver fibrosis, myocardial infarction, shock, stroke, vasculitis, sepsis, inflammatory bowel disease, systemic lupus erythematosus or atopic dermatitis. The concentration or amount of the pharmaceutical composition, the types of the carriers, and other properties are mostly the same as those of the pharmaceutical composition described above, and therefore is omitted here for conciseness.

As mentioned above, the pharmaceutical composition of this invention and use thereof can inhibit PI3K activity in a patient for avoiding the effect of PI3K to the function of the immune system, and thus achieves the efficacy of treating diseases.

### Example 1

### The Results of Palbociclib Inhibiting PI3K Activity

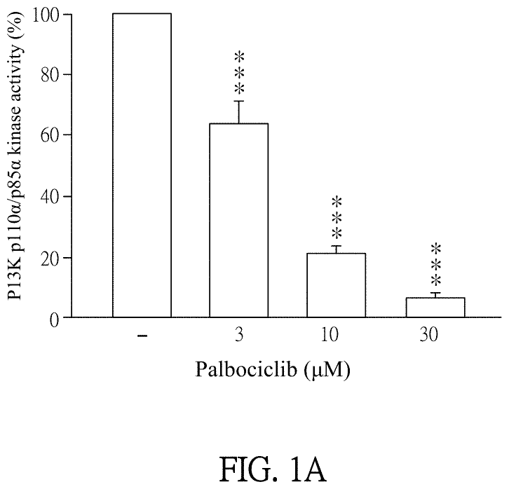

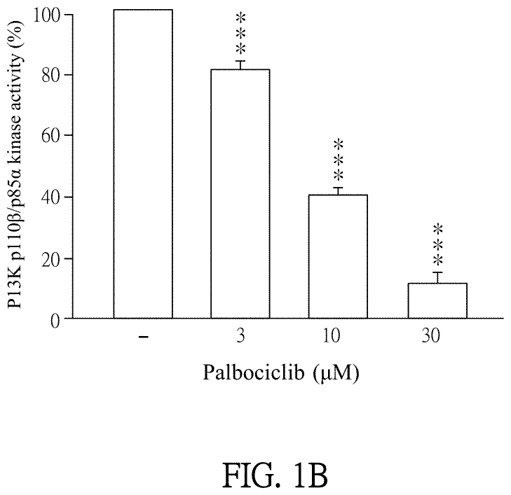

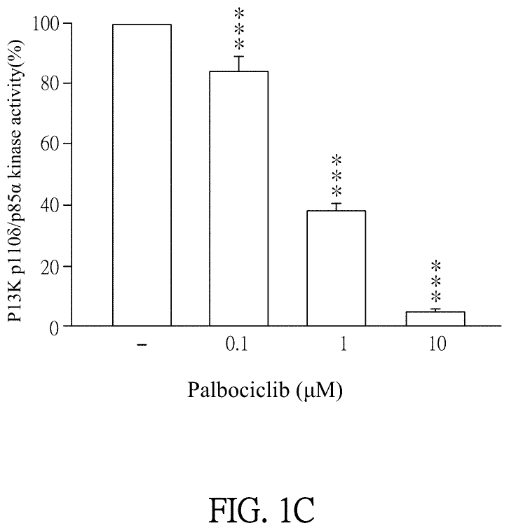

Palbociclib was dissolved in ddH₂O (as solvent) and then added to the reagents which contain PI3K p110α/p85α (50 ng/ml), PI3K p110β/p85α (150 ng/ml), or PI3K p110δ/p85α (50 ng/ml). The final concentration of Palbociclib was 0.1 to 30 μM. Then, the mixtures were incubated for 10 minutes at 25° C. The reagent which was added by ddH₂O (solvent, marked as “−” in FIGS. 1A-1C) instead of Palbociclib was as a control in this experiment. 2.5X ATP/substrate was added to each reagent and then further incubated for 60 minutes. Finally, enzymatic activities of PI3K were analyzed according to the user manual of Kinase-Glo® Luminescent Kinase Assays (Promega Corporation, catalog number V1690) and luminometer (TECAN infinite 200 Pro) by using Kinase-Glo® Luminescent Kinase Assays. The results were shown in FIGS. 1A to 1C.

Please refer to FIGS. 1A to 1C. FIG. 1A shows a result of PI3K-α activity inhibited by the different amount of Palbociclib. FIG. 1B shows a result of PI3K-rβ activity inhibited by the different amount of Palbociclib. FIG. 1C shows a result of PI3K-δ activity inhibited by the different amount of Palbociclib. As shown in FIGS. 1A-1C, Palbociclib significantly inhibits PI3K activity except the control (marked as “−”). In more detailed, as shown in FIG. 1A, 3 μM, 10 μM and 30 μM of Palbociclib significantly inhibit PI3K p110α/p85α activity. PI3K p110α/p85α activity is significantly decreased along with increasing the concentration of Palbociclib. As shown in FIG. 1B, 3 μM, 10 μM and 30 μM of Palbociclib significantly inhibit PI3K p110β/p85α activity. PI3K p110β/p85α activity is significantly decreased along with the increase of concentration of Palbociclib. As shown in FIG. 1C, 0.1 μM, 1 μM and 10 μM of Palbociclib significantly inhibit PI3K p110δ/p85α activity. PI3K p110δ/p85α activity is significantly decreased along with increasing the concentration of Palbociclib. As shown in FIGS. 1A-1C, “−” is control; *** is p<0.001 compared with control. All data are expressed as mean±standard error of the mean (SEM) (n=6); error bar presents SEM.

### Example 2

### Palbociclib Restricts Superoxide Anion Generation and Elastase Release in the fMLF-Stimulated Neutrophils

### Donors:

Donors were healthy individuals aged between 20 to 35 years old who have no any known diseases or risk factors and had normal work-rest cycles and did not take medicine for more than one week. The informed consent to participate which was approved by the Institutional Review Board are signed by the donors. 50 mL of blood was collected from the median cubital vein of each donor by sterile vacutainer, and was used to isolate neutrophils for the following experiments.

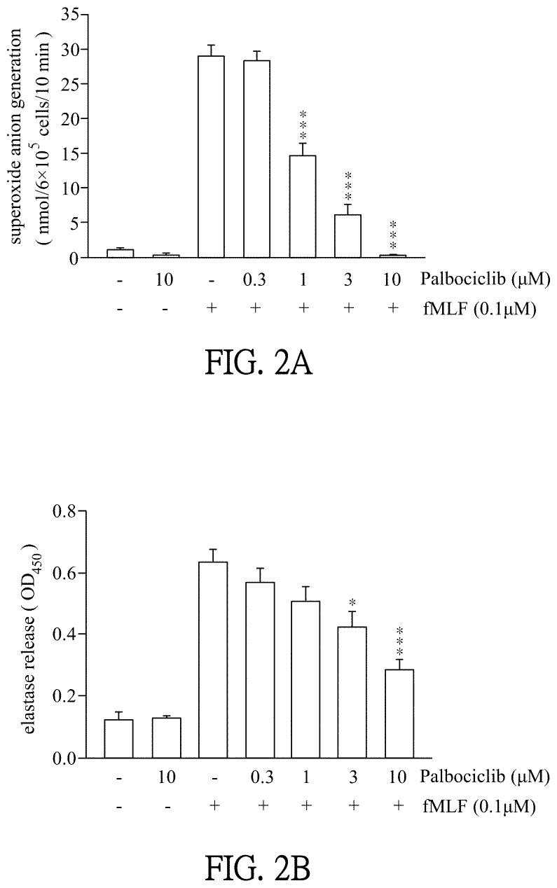

Human neutrophils (isolated from whole blood of donors) were incubated with ddH₂O (as solvent, marked as “−” in the groups at X-axis in FIGS. 2A and 2B) or Palbociclib (0.3-10 μM, dissolved in ddH₂O) for 5 minutes, and then stimulated the neutrophils with or without fMLF (0.1 μM)/cytochalasin B (CB, 1 μg/ml) for another 10 minutes to activate the neutrophils. The absorbances (OD₅₅₀ and OD₄₅₀) of the reaction mixture were then measured by a spectrophotometer at OD 550 nm and OD 450 nm. The reading of OD₅₅₀ is used to detect the reduction of ferricytochrome c, which indicates the amount of superoxide anion. The reading of OD₄₅₀ is used to detect the status of Methoxysuccinyl-Ala-Ala-Val-p-nitroanilide (which is a specific substrate of elastase) affected by elastase, which indicates the release amount and activity of elastase. Cytochalasin B was sold by Sigma-Aldrich, catalog number #2506; fMLF was sold by Sigma-Aldrich, catalog number #454454; model number of the spectrophotometer is HITACHI U-3010. The increased amount of superoxide anion and the increased activity and release amount of elastase indicate that the activity of neutrophils is higher.

Please refer to FIGS. 2A-2B, FIG. 2A shows a result that Palbociclib significantly restricts superoxide anion generation in the fMLF-stimulated neutrophils. FIG. 2B shows a result that Palbociclib significantly restricts elastase release in the fMLF-stimulated neutrophils. As shown in FIG. 2A, the group which is incubated with ddH₂O (instead of Palbociclib) and then stimulated with fMLF is played as the control in this experiment. Compared with the control, the group which is incubated with Palbociclib and then stimulated by fMLF can restrict the generation of superoxide anion, and the groups of adding 1 μM, 3 μM and 10 μM of Palbociclib significantly restrict the generation of superoxide anion. Moreover, the generation of superoxide anion is significantly decreased along with increasing the concentration of Palbociclib. As shown in FIG. 2B, the group which is incubated with ddH₂O (instead of Palbociclib) and then stimulated with fMLF is played as the control in this experiment. Compared with the control, the group which is incubated with Palbociclib and then stimulated by fMLF can reduce the release of elastase, and the groups of adding 3 μM and 10 μM of Palbociclib significantly restrict the release of elastase. Moreover, the release of elastase is significantly decreased along with increasing the concentration of Palbociclib. As shown in FIGS. 2A-2B, “−” is without Palbociclib (added ddH₂O); “+” is added; *p<0.05 compared with the control; ***p<0.001 compared with the control. All data are expressed as mean±SEM (n=6); error bar presents SEM.

### Example 3

### Preparation of the Pharmaceutical Composition

500 μg of Palbociclib was dissolved in 100 μl of 60% ethanol to make a pharmaceutical composition. The Palbociclib concentration of the pharmaceutical composition was 500 μg/100 μl and used in the subsequent animal experimental example 4.

### Example 4

### Palbociclib Reducing the Psoriasis-Like Symptoms in the IMQ-Treated Mice

Male BALB/c mice (7-9 weeks of age, the body weight of each mouse is about 20 g) were divided into different groups, and each group has 6 mice. Each mouse was epilation on the back and then treated with 100 μl of solvent (60% ethanol) or 100 μl of the pharmaceutical composition prepared in example 3 (the concentration of Palbociclib is 25 mg/kg of body weight) by applying on the nude back skin before topical treatment of imiquimod (IMQ, sold by 3M Health Care Limited). And then applying with or without IMQ on the nude back skin of mice to cause psoriasis-like symptom. Each treatment was given once per day for 5 consecutive days. The mice which applying solvent and IMQ is played as the control. The mice which merely applying solvent or Palbociclib without IMQ are played as the blank 1 and blank 2.

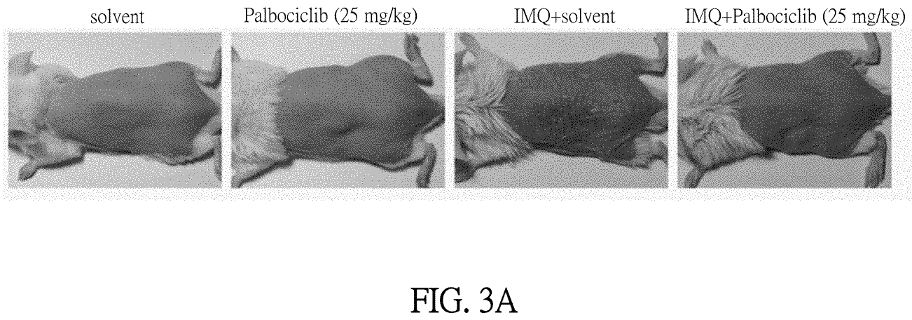

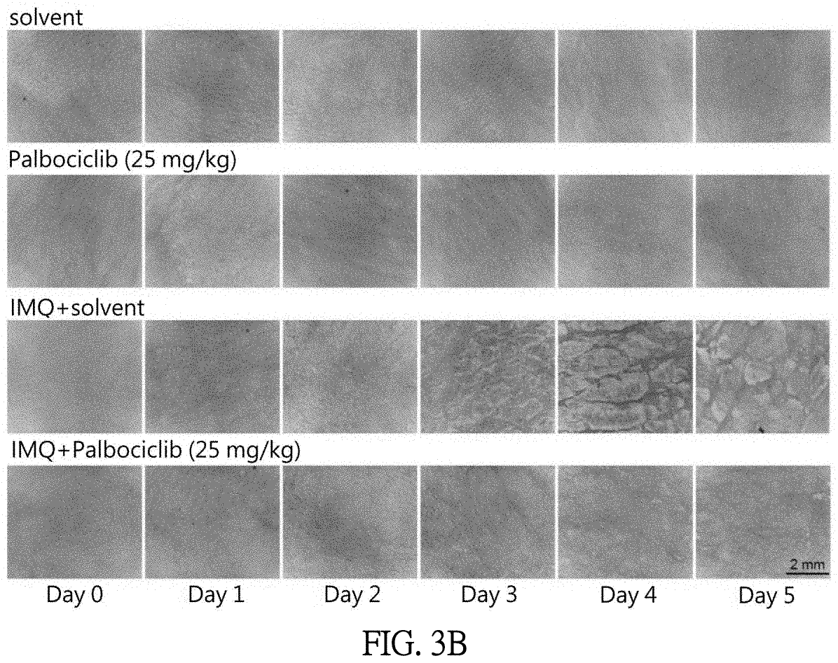

Please refer to FIGS. 3A-3B, FIG. 3A shows the photos of mice skin after 5^{th} day treated by different treatments. FIG. 3B shows the partially enlarged photos of mice skin after 0 to 5^{th} day treated by different treatments. The photos of FIG. 3B were taken by handheld digital microscope. As shown in FIG. 3A, the group merely treated by solvent or Palbociclib without IMQ did not cause psoriasis-like symptoms during 5 days after the treatments. The control group treated by solvent and then IMQ cause psoriasis-like symptoms. Compared with the control, the psoriasis-like symptoms significantly improved in the group treated by Palbociclib and then IMQ. As shown in FIG. 3B, the group merely treated by solvent or Palbociclib without IMQ did not cause psoriasis-like symptoms during 5 days after the treatments. The control group treated by solvent and IMQ start to cause psoriasis-like symptoms after the treatment in 3^{rd} day. Compared with the control, the psoriasis-like symptoms were not observed in the group treated by Palbociclib and IMQ after the treatment in 5^{th} day. The results of this example indicate that Palbociclib can improve or cure the psoriasis-like symptom.

According to the result of example 1, the pharmaceutical composition of this invention may significantly inhibits PI3K activity (including PI3K-α, PI3K-β and PI3K-δ). According to the results of example 2, the pharmaceutical composition of this invention may significantly inhibit neutrophils activity. According to the result of example 4, the pharmaceutical composition of this invention may improve or cure the psoriasis-like symptoms. The examples are described for illustration but not intended to be limiting.

As mentioned above, the pharmaceutical composition and use of this invention can inhibit PI3K activity and neutrophils activity in a patient for avoiding the effect of PI3K and neutrophils to the function of the immune system, and thus achieves the efficacy of treating diseases.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A pharmaceutical composition for treating a disease in a patient, the pharmaceutical composition comprises an effective amount of Palbociclib and a pharmaceutically acceptable carrier, wherein Palbociclib is used to treat the disease by inhibiting phosphatidylinositol-4,5-bisphosphate 3-kinase (PI3K) activity.

2. The pharmaceutical composition according to claim 1, wherein the disease is acute lung injury, acute respiratory distress syndrome, psoriasis, chronic obstructive pulmonary disease, pulmonary fibrosis, liver injury, fatty liver disease, liver fibrosis, myocardial infarction, shock, stroke, vasculitis, sepsis, inflammatory bowel disease, systemic lupus erythematosus or atopic dermatitis.

3. The pharmaceutical composition according to claim 1, wherein Palbociclib is used to treat the disease by inhibiting neutrophils activity.

4. The pharmaceutical composition according to claim 1, wherein the effective amount of Palbociclib is from 1 μg/kg of the body weight/per day to 100 mg/kg of the body weight/per day.

5. A pharmaceutical composition for use in the treatment of a disease in a patient, wherein the pharmaceutical composition comprises an effective amount of Palbociclib and a pharmaceutically acceptable carrier, Palbociclib is used to treat the disease by inhibiting PI3K activity.

6. The pharmaceutical composition for use according to claim 5, wherein the disease is acute lung injury, acute respiratory distress syndrome, psoriasis, chronic obstructive pulmonary disease, pulmonary fibrosis, liver injury, fatty liver disease, liver fibrosis, myocardial infarction, shock, stroke, vasculitis, sepsis, inflammatory bowel disease, systemic lupus erythematosus or atopic dermatitis.

7. The pharmaceutical composition for use according to claim 5, wherein Palbociclib is used to treat the disease by inhibiting neutrophils activity.

8. The pharmaceutical composition for use according to claim 5, wherein the effective amount of Palbociclib is from 1 μg/kg of the body weight/per day to 100 mg/kg of the body weight/per day.

9. A pharmaceutical composition for inhibiting PI3K activity, the pharmaceutical composition comprises an effective amount of Palbociclib and a pharmaceutically acceptable carrier.

10. The pharmaceutical composition according to claim 9, wherein the pharmaceutical composition further inhibits neutrophils activity.

11. The pharmaceutical composition according to claim 9, wherein the effective amount of Palbociclib is from 1 μg/kg of the body weight/per day to 100 mg/kg of the body weight/per day.

12. A method for treating a disease in a patient, the method comprises a step of providing a pharmaceutical composition to the patient, the pharmaceutical composition comprises an effective amount of Palbociclib and a pharmaceutically acceptable carrier, wherein Palbociclib is used to treat the disease by inhibiting PI3K activity.

13. The method according to claim 12, wherein the disease is acute lung injury, acute respiratory distress syndrome, psoriasis, chronic obstructive pulmonary disease, pulmonary fibrosis, liver injury, fatty liver disease, liver fibrosis, myocardial infarction, shock, stroke, vasculitis, sepsis, inflammatory bowel disease, systemic lupus erythematosus or atopic dermatitis.

14. The method according to claim 12, wherein Palbociclib is used to treat the disease by inhibiting neutrophils activity.

15. The method according to claim 12, wherein the effective amount of Palbociclib is from 1 μg/kg of the body weight/per day to 100 mg/kg of the body weight/per day.

16. A method for inhibiting PI3K activity in a patient, the method comprises a step of providing a pharmaceutical composition to the patient, the pharmaceutical composition comprises an effective amount of Palbociclib and a pharmaceutically acceptable carrier.

17. The method according to claim 16, wherein Palbociclib further inhibits neutrophils activity.

18. The method according to claim 16, wherein the effective amount of Palbociclib is from 1 μg/kg of the body weight/per day to 100 mg/kg of the body weight/per day.
